(19) United States
(12) Patent Application Publication
(10) Pub. No.: US 2017/0273811 A1
(43) Pub. Date: Sep. 28, 2017

(54) STENT

(71) Applicant: TERUMO KABUSHIKI KAISHA, Tokyo (JP)

(72) Inventors: Ryouta IKEUCHI, Shizuoka (JP); Miho MUKADA, Shizuoka (JP)

(73) Assignee: TERUMO KABUSHIKI KAISHA, Tokyo (JP)

(21) Appl. No.: 15/470,227

(22) Filed: Mar. 27, 2017

(30) Foreign Application Priority Data
Mar. 28, 2016 (JP) 2016-064174

(51) Int. Cl. A61F 2/915; A61F 2/95; A61F 2/92

(52) U.S. Cl. CPC A61F 2/915; A61F 2/92; A61F 2/95; A61F 2/82

(57) ABSTRACT

A stent is configured so that the length during expansion is unlikely reduced and has satisfactory flexibility so as to be able to pass through a biological lumen. In the stent, the number of mountains in a first annular portion and the number of mountains in a second annular portion between the first link portions adjacent to each other in a circumferential direction differ from each other. The first link portions are positioned on a first line parallel to an axial direction and the second link portions are positioned on a second line parallel to the axial direction, in a state where a plurality of annular reference bodies are repeatedly arranged in the axial direction.

### CROSS-REFERENCES TO RELATED APPLICATIONS

This application claims priority to Japanese Application No. 2016-064174 filed on Mar. 28, 2016, the entire content of which is incorporated herein by reference.

### TECHNICAL FIELD

The present generally invention relates to a stent.

### BACKGROUND DISCUSSION

A stent is a device which maintains a patency state of a blood vessel after being indwelled in, for example, a stenosed portion or a closed portion, which is present in the blood vessel, in an expanded state. A cylindrical circumference in which gaps are formed is formed in a stent using a linear strut. An example of such a stent is disclosed in International Patent Application Publication No. 2007/040250.

### SUMMARY

A stent of which the length during expansion is unlikely reduced and which has satisfactory flexibility so as to be able to pass through a biological lumen is required, in order to accurately indwell the stent in a target lesion area.

However, in reality, there is no stent that is well suited to accomplishing such results.

A stent is disclosed which has satisfactory flexibility so as to be able to pass through a biological lumen and has a length during expansion that is unlikely reduced, and therefore, it is possible to accurately indwell the stent in a target lesion area.

According to one aspect, a stent comprises: a plurality of struts which together form a cylindrical circumference, with each strut possessing a wave shape in which a plurality of mountains are configured as convex portions that project toward both ends of the cylindrical shape in an axial direction; and annular bodies repeatedly arranged in the axial direction, with each annular body including at least a first annular portion comprised of one of the struts and possessing an annular shape and a second annular portion comprised of another of the struts and possessing an annular shape, the second annular portion being positioned adjacent the first annular portion in the axial direction. A plurality of first link portions are disposed between the first annular portion and the second annular portion of each of the annular bodies, with the first link portions of each annular body being circumferentially spaced from each other and connecting the first annular portion to the second annular portion. A plurality of second link portions are disposed between and connecting the annular bodies that are adjacent to each other in the axial direction, with the second link portions connecting the axially adjacent annular bodies being circumferentially spaced from each other. Intervals between the first link portions adjacent to each other in the circumferential direction are equal, and intervals between the second link portions adjacent to each other in the circumferential direction are equal. For each of the annular bodies, a total number of mountains in the first annular portion and a total number of mountains in the second annular portion between the circumferentially adjacent first link portions differing from each other. One of the first link portions of each annular body is positioned on one first line parallel to the axial direction, the other of the first link portions of each annular body is positioned on another first line different from the one first line and parallel to the axial direction, and one of the second link portions between each pair of axially adjacent annular bodies is positioned on a second line parallel to the axial direction.

According to the stent constituted as described above, the first annular portion in which the strut “densely” exists and the second annular portion in which the strut “sparsely” exists are regularly repeatedly arranged in the axial direction. Furthermore, a first link portion is coaxially positioned (on a first line) and a second link portion is coaxially positioned (on a second line). For this reason, the stent becomes a stent of which an expansion state and an expansion timing become uniform in the axial direction and the length during expansion is unlikely reduced and which has satisfactory flexibility so as to be able to pass through a biological lumen.

A stent according to another aspect possesses a central axis and an axial extent, and comprises: a plurality of annular bodies arranged in an axial direction along the axial extent to define a cylindrical shape with a plurality of through openings, with each annular body comprising: a first annular portion comprised of a strut possessing an annular shape; and a second annular portion comprised of a strut possessing an annular shape, the second annular portion being positioned axially adjacent the first annular portion. The strut comprising the first annular portion of each annular body is wavy-shaped in which a plurality of mountains project towards opposite axial ends of the stent, and the strut comprising the second annular portion of each annular body is wavy-shaped in which a plurality of mountains project towards the opposite axial ends of the stent. Each of the annular bodies comprises a plurality of circumferentially spaced apart first link portions connecting the strut of the first annular portion and the strut of the second annular portion, with the circumferentially spaced apart first link portions of each annular body including one first link portion and an other first link portion. Each pair of axially adjacent annular bodies is connected to one another by at least one second link portion, and the plurality of annular bodies comprise a first annular body, a second annular body and a third annular body. The first link portions of the first annular body is circumferentially spaced apart by a circumferential spacing that is the same as the circumferential spacing between the first link portions of the second annular body and that is also the same as the circumferential spacing between the first link portions of the third annular body. For each of the first, second and third annular bodies, the total length of the strut of the first annular portion and the total length of the strut of the second annular portion differ from each other, with the total length of the strut of the first annular portion being defined by the distance from a point on the strut of the first annular portion along the entire circumference of the strut of the first annular portion back to the point on the strut of the first annular portion, and the total length of the strut of the second annular portion being defined by the distance from a point on the strut of the second annular portion along the entire circumference of the strut of the second annular portion back to the point on the strut of the second annular portion. The one first link portion of the first annular body, the one first link portion of the second annular body and the one first link portion of the third annular body are aligned with one another and positioned along one first line parallel to the central axis. The other first link portion of the first annular body, the other first link portion of the second annular body and the other first link portion of the third annular body are aligned with one another and are positioned along an other first line parallel to the central axis and circumferentially spaced from the one first line. The second link portion of the first annular body, the second link portion of the second annular body and the second link portion of the third annular body are aligned with one another and are positioned along a second line parallel to the central axis and circumferentially spaced from the one first line and the other first line.

In accordance with another aspect, a method comprises: inserting a stent into a living body, with the stent being positioned on a balloon and comprising: a plurality of annular bodies arranged in an axial direction along the axial extent to define a cylindrical shape with a plurality of through openings, each annular body comprising a first annular portion comprised of a strut possessing an annular shape and a second annular portion comprised of a strut possessing an annular shape, the second annular portion being positioned axially adjacent the first annular portion; the strut comprising the first annular portion of each annular body and the strut comprising the second annular portion of each annular body being wavy-shaped in which a plurality of mountains project towards opposite axial ends of the stent, the strut comprising the first annular portion of each annular body being more dense than the strut comprising the second annular portion of each annular body. The method also comprises inflating the balloon to outwardly expand the second annular portion of the stent more easily than the first annular portion of the stent by virtue of the strut comprising the first annular portion of each annular body being more dense than the strut comprising the second annular portion of each annular body.

### BRIEF DESCRIPTION OF THE DRAWINGS

FIG. 1 is a perspective view showing a balloon catheter on which a stent is mounted.

FIG. 2 is a perspective view showing an enlarged distal portion of the balloon catheter shown in FIG. 1.

FIG. 3 is a development view, in which a part of the circumference of the stent is cut into a linear shape and is developed along an axial direction, and is a development view before the stent is expanded.

FIG. 4 is a partially enlarged view of FIG. 3.

FIG. 5 is a development view, in which a part of the circumference of the stent is cut into a linear shape and is developed along the axial direction, and is a development view when the stent is expanded.

### DETAILED DESCRIPTION

Hereinafter, an embodiment of the disclosed stent, representing an example of the inventive stent disclosed here, will be described with reference to the accompanying drawings. Note that the following description does not restrict the scope of the invention defined in the claims or the definition of terms. In addition, in some cases, dimensional ratios in the drawings are exaggerated and are different from the actual ratios for the convenience of description and to facilitate an understanding. In addition, in the description below, the hand operation side of a medical device will be referred to as a “proximal side” or “proximal end”, and the side through which the medical device is inserted into the biological lumen will be referred to as a “distal side” or “distal end”.

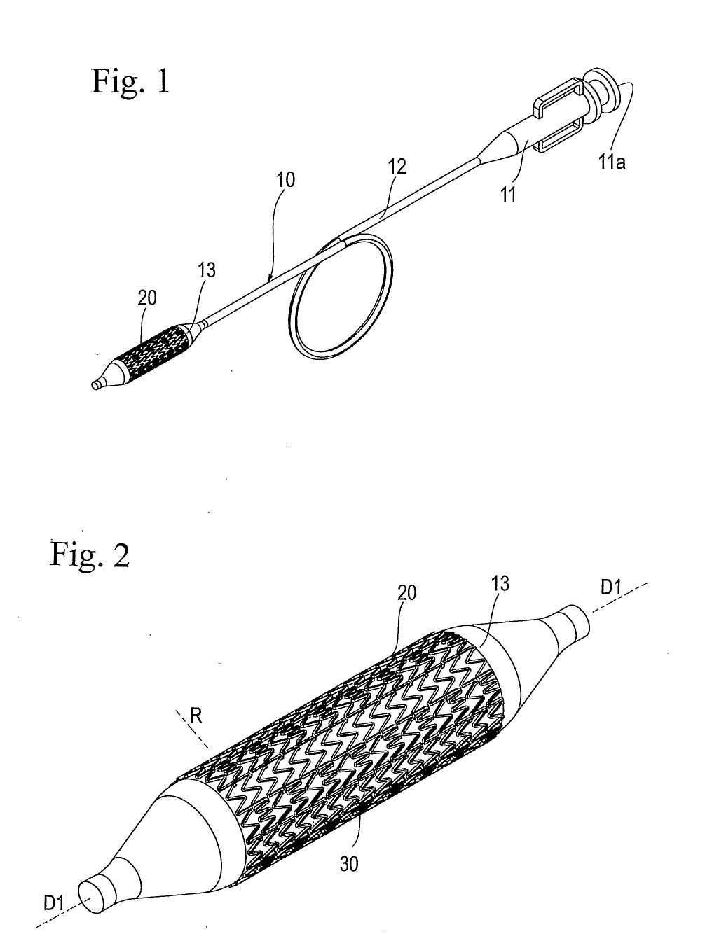

FIG. 1 is a perspective view showing a balloon catheter 10 on which a stent 20 is mounted and FIG. 2 is a perspective view showing an enlarged distal portion of the balloon catheter 10 shown in FIG. 1.

Referring to FIG. 1, the balloon catheter 10 has an elongated shaft portion 12, a hub 11 disposed in a proximal portion of the shaft portion 12, and a dilatable balloon 13 disposed in a distal portion of the shaft portion 12. Referring to FIG. 2, the stent 20 has a cylindrical shape and is mounted on the balloon 13. The stent 20 includes a plurality of struts 30 which form a cylindrical circumference. As shown in the illustrated embodiment, the struts may be relatively thin members.

The hub 11 has an interlock port 11a configured to interlock or engage with an inflator for supplying a fluid to the balloon 13 to inflate and dilate the balloon 13. Examples of the dilation fluid for dilating the balloon 13 include an X-ray contrast agent, a physiological salt solution, and an electrolytic solution, though the dilation fluid is not limited to those listed.

The shaft portion 12 has a lumen which allows communication between the interlock port 11a of the hub 11 and the inside of the balloon 13.

The balloon 13 is fixed to the circumference of the shaft portion 12 in the vicinity of the distal portion and is disposed in a state of being folded (or a state of being deflated). The inside of the balloon 13 communicates with the interlock port 11a of the hub 11 via the lumen in the shaft portion 12. A dilation fluid which has been made to flow in from the interlock port 11a of the hub 11 flows into the balloon 13. The balloon 13 is dilated due to the dilation fluid which has been introduced.

The constituent material of the balloon 13 preferably has a flexibility, and examples of the material for fabricating the balloon 13 include polymer materials such as polyolefins, a cross-linked body of polyolefins, polyester, polyester elastomer, polyvinyl chloride, polyurethane, polyurethane elastomer, polyphenylene sulfide, polyamide, polyamide elastomer, and fluororesin, silicone rubber, and latex rubber. Polyester is, for example, polyethylene terephthalate. The constituent material of the balloon 13 is not limited to a form of singly using the above-described polymer material, and it is also possible to apply, for example, a film on which the above-described polymer material is appropriately stacked.

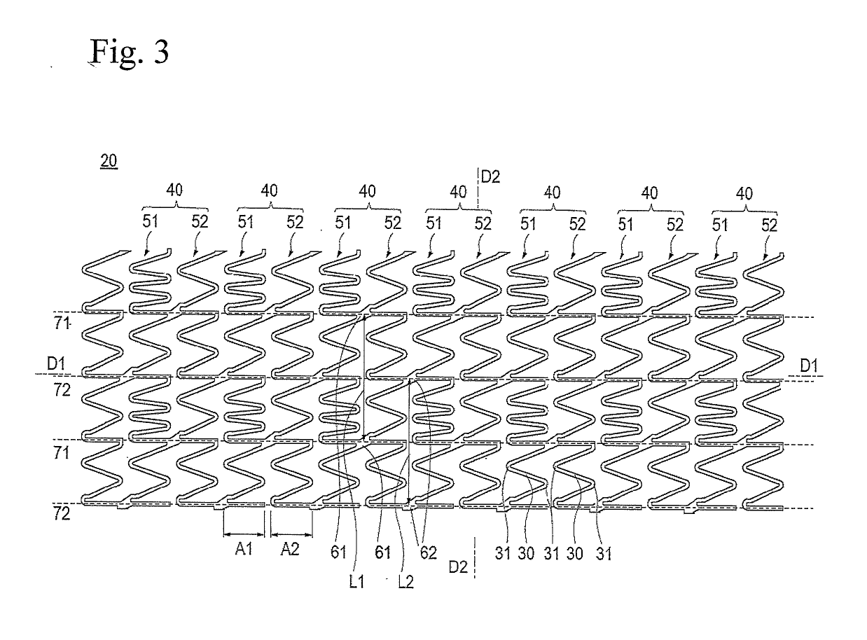

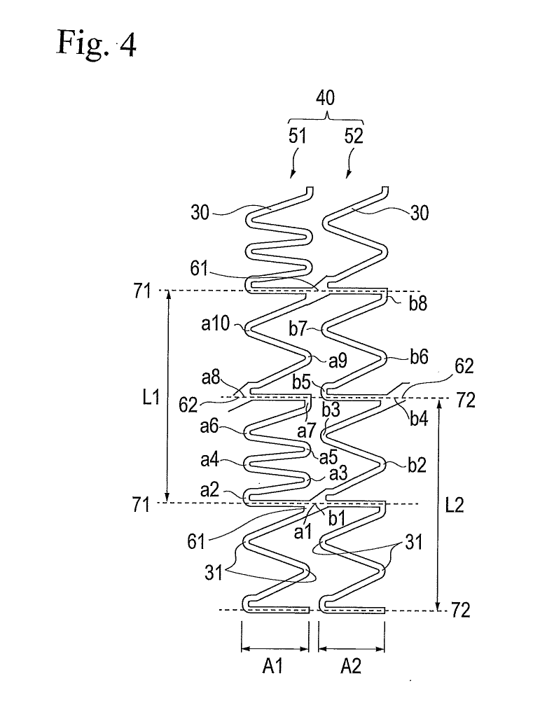

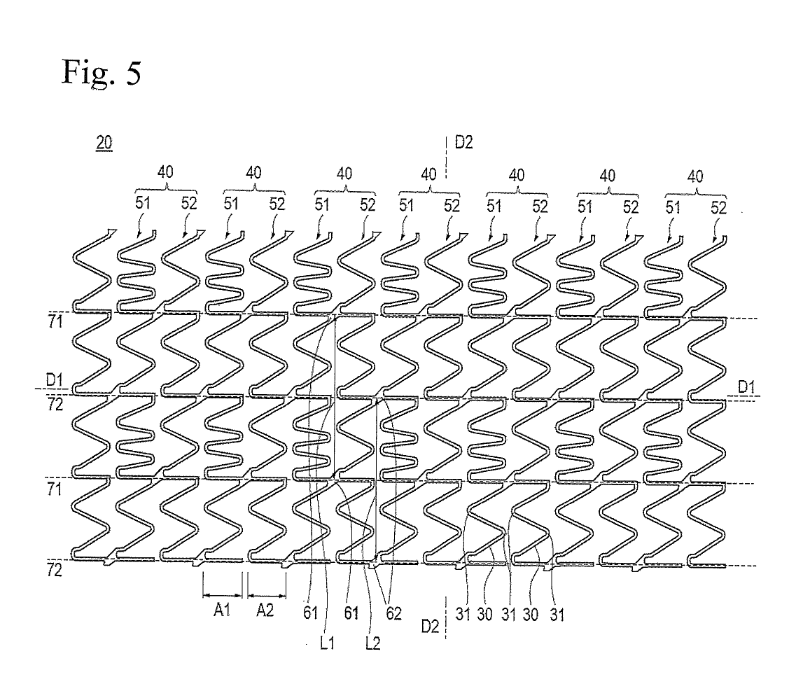

FIG. 3 is a development view of the stent, in which a part of the circumference of the stent 20 is cut into a linear shape and is developed along an axial direction, and is a development view before the stent 20 is expanded. FIG. 4 is a partially enlarged view of a portion of the stent shown in FIG. 3. FIG. 5 is a development view, in which a part of the circumference of the stent 20 is cut into a linear shape and is developed along the axial direction, and is a development view when the stent 20 is expanded.

Referring to FIGS. 3 to 5, the stent 20 of the embodiment has the struts 30 which may be relatively thin components. In the present specification, the axial direction of the cylindrical shape formed by the struts 30 is denoted as an “axial direction D1” (refer to FIGS. 2, 3, and 5), the circumferential direction of the cylindrical shape is denoted as a “circumferential direction D2” (refer to FIGS. 3 and 5), and the radial direction of the cylindrical shape is denoted as a “radial direction R” (refer to FIG. 2).

The struts 30 form the circumference of the cylindrical shape in which gaps (through holes or through openings) are formed, as shown in FIG. 2. In addition, the struts 30 each extend in the circumferential direction D2 while being folded back in a wave (zig-zag) shape, and each form an endless tubular shape.

Referring to FIGS. 3 to 5, the struts 30 have a wave shape in which a plurality of mountains 31 (or peaks) as convex portions are formed toward both sides or ends (both the right and left sides or both axial ends in the FIG. 3) in the axial direction D1.

The stent 20 of the embodiment has annular bodies 40 which are units repeatedly arranged in the axial direction D1. In the illustrated example, seven annular bodies 40 are arranged in the axial direction D1.

Each annular body 40 includes a first annular portion 51 which is comprised of one of the struts 30 in an annular shape, and a second annular portion 52 which is comprised of one of the struts 30 in an annular shape and adjacent to the first annular portion 51 in the axial direction D1. As shown in FIGS. 3 and 5, in the illustrated embodiment, each of the annular bodies 40 is configured so that the first annular portion 51 of one annular body 40 is always positioned axially between the second annular portion 52 of the same annular body and the second annular portion 52 of the axially adjacent annular body 40. The annular body 40 further has a plurality of first link portions 61 (two in the present embodiment) which are disposed in the circumferential direction D2 between the first annular portion 51 and the second annular portion 52 adjacent to each other in the axial direction D1. The first link portion 61 connects the axially adjacent first annular portion 51 and the second annular portion 52 to each other.

The stent 20 has a plurality of second link portions 62 (two in the present embodiment) which are disposed in the circumferential direction D2 between the annular bodies 40 adjacent to each other in the axial direction D1. The second link portion 62 connects the axially adjacent annular bodies 40 to each other.

The first annular portion 51 and the second annular portion 52 in each annular body 40 are connected by the first link portions 61, and the second annular portion 52 and the first annular portion 51 which are axially adjacent to each other and are included in the axially adjacent annular bodies 40 are connected to each other by the second link portion 62.

An interval (L1) between the first link portions 61 adjacent to each other in the circumferential direction D2 is the same in each of the annular bodies 40. In addition, an interval (L2) between the second link portions 62 adjacent to each other in the circumferential direction D2 is equal.

The number (total number) of mountains 31 in the first annular portion 51 and the number (total number) of mountains 31 in the second annular portion 52 between the circumferentially adjacent first link portions 61 (i.e., the first link portions 61 that are adjacent to each other in the circumferential direction D2) differ from each other. As shown in FIG. 4, the number of mountains 31 in the first annular portion 51 is ten (a1 to a10) and the number of mountains 31 in the second annular portion 52 is eight (b1 to b8). Regarding the mountains 31 in the first annular portion 51, mountains 31 (a1, a3, a5, a7, and a9) are convex portions toward a right side in the drawing in the axial direction D1 and mountains 31 (a2, a4, a6, a8, and a10) are convex portions toward a left side in the drawing in the axial direction D1. Regarding the mountains 31 in the second annular portion 52, mountains 31 (b2, b4, b6, and b8) are convex portions toward a right side in the drawing in the axial direction D1 and mountains 31 (b1, b3, b5, and b7) are convex portions toward a left side in the drawing in the axial direction D1. The distance, that is, the amplitude between the mountains 31 (a1, a3, a5, a7, and a9) and the mountains 31 (a2, a4, a6, a8, and a10) in the first annular portion 51 in the axial direction D1 is represented or designated by A1. The distance, that is, the amplitude between the mountains 31 (b2, b4, b6, and b8) and the mountains 31 (b1, b3, b5, and b7) in the second annular portion 52 in the axial direction D1 is represented or designated by A2. The amplitude A1 of the first annular portion 51 is equal to the amplitude A2 of the second annular portion 52.

The number of mountains 31 (ten (a1 to a10)) in the first annular portion 51 is greater than the number of mountains 31 (eight (b1 to b8)) in the second annular portion 52. In addition, the amplitude A1 of the first annular portion 51 is equal to the amplitude A2 of the second annular portion 52. Accordingly, the total length of the strut 30 in the first annular portion 51 is longer than the total length of the strut 30 in the second annular portion 52. The total length of the strut of the first or second annular portion refers to the distance (distance along a centerline of the strut) from a point on the strut along the entire circumference of the strut and back to the point on the strut. In other words, the strut 30 of the first annular portion 51 “densely” exists relative to, or is more dense than, the strut 30 of the second annular portion 52. In addition, it can be said that the strut 30 of the second annular portion 52 “sparsely” exists relative to, or is less dense than, the strut 30 of the first annular portion 51. The amount of pinching of the balloon 13 by the strut 30 during crimping is larger in the second annular portion 52 in which the strut 30 “sparsely” exists than in the first annular portion 51 in which the strut 30 “densely” exists. Pinching during crimping refers to portions of the balloon 13 that are caught between parts of the struts 30 when the stent 20 is placed on the balloon and is reduced in outer diameter. Accordingly, when the balloon 13 is dilated, the second annular portion 52 of the stent 20 is more easily expanded than the first annular portion 51.

As shown in FIGS. 3 and 5, the first link portions 61 are positioned on first lines 71 parallel to the axial direction D1 and the second link portions 62 are positioned on the second lines 72 parallel to the axial direction D1, in a state where the plurality of annular bodies 40 are repeatedly arranged in the axial direction D1. In terms of the first link portions 61, one first line 71 passes through one of the first link portions 61 of each annular body 40, and another first line 71 passes through the other first link portion 61 of each annular body 40 as shown in FIGS. 3 and 5. Similarly, in terms of the second link portions 62, one second line 72 passes through one of the second link portions 62 of each annular body 40, and another second line 72 passes through the other second link portion 62 of each annular body 40 as shown in FIGS. 3 and 5. It is sufficient for the “first line 71” and the “second line 72” to have substantially linear shape, and it is understood that a small curved portion or a case of partially having a small bent portion is also included.

Since the first link portions 61 are coaxially positioned (on the first lines 71) and the second link portions 62 are coaxially positioned (on the second lines 72), it is possible to secure the collapse strength in the axial direction D1, and therefore, the length variation (shortening) of the stent 20 during expansion in the axial direction D1 becomes almost zero. In addition, since the stent 20 has a relatively high strength in the axial direction D1, it is difficult for the stent 20 to be deformed (deformation) in the axial direction D1 when delivering the stent 20 using the balloon catheter 10.

It is preferable that the first lines 71 on which the first link portions 61 are positioned and the second lines 72 on which the second link portions 62 are positioned are offset to each other in the circumferential direction D2. That is, the two first lines 71, 71 and the two second lines 72 are each circumferentially spaced apart from one another.

When comparing the case with a case where the first lines 71 and the second lines 72 are coaxially positioned together, twisting of the whole stent 20 in the circumferential direction D2 or deflection from the axial direction D1 is more easily caused, and therefore, the flexibility of the stent 20 is improved. As a result, it is possible to obtain more satisfactory passing properties of the stent 20 at a position of a curved lesion or the like.

The number of first link portions 61 in one annular body 40 and the number of second link portions 62 between axially adjacent annular bodies 40 are equal to each other. Furthermore, the plurality of second link portions 62 preferably connect the annular bodies 40 at an axially central position (in the present embodiment, a position which is at 90° (offset at a rotational angle of 90°) to the first link portion 61 in the circumferential direction D2) between the first link portions 61 adjacent to each other in the circumferential direction D2.

An interval (L1) between the first link portions 61 adjacent to each other in the circumferential direction D2 and an interval (L2) between the second link portions 62 adjacent to each other in the circumferential direction D2 are equal to each other, and the first line 71 at which the first link portion 61 is positioned and the second line 72 at which the second link portion 62 is positioned are offset while being evenly spaced in the circumferential direction D2. Accordingly, it is possible to uniformly secure the collapse strength of the stent 20 in the axial direction D1 in the circumferential direction D2 and to uniformly secure the flexibility of the stent 20 regardless of the position of the axial direction D1. As a result, it is possible to obtain more satisfactory passing properties of the stent 20 at a position of a curved lesion or the like while uniformly maintaining the high strength in the axial direction D1, in the circumferential direction D2.

The stent 20 functions as an in-vivo indwelling object which holds the lumen at an appropriate size by being indwelled in a stenosed site which is a lesion area by being closely adhered to the inner surface thereof. The stent 20 has a cylindrical shape and is expanded in accordance with the dilation of the balloon 13. The stent 20 is, for example, a drug eluting stent 20 (DES), in which the outer surface is coated with a drug.

The stent 20 is formed of a material having biocompatibility. Examples of the material having biocompatibility include iron, titanium, aluminum, tin, tantalum or tantalum alloy, platinum or platinum alloy, gold or gold alloy, titanium alloy, nickel-titanium alloy, cobalt-based alloy, cobalt-chromium alloy, stainless steel, zinc-tungsten alloy, and niobium alloy.

A drug with which the outer surface of the stent 20 is coated is a mixture of a biologically/physiologically active substance and a biodegradable polymer. Note that the area to be coated with the drug is not limited to the outer surface of the stent 20, and may be the inner surface of the stent 20, or both of the outer surface and the inner surface.

A method for manufacturing the stent 20 is not particularly limited, but examples include a method for cutting a stent from a tube formed of the above-described material using laser or the like, a method performed through injection molding, and a method for performing stacking (lamination molding) using a 3D printer or the like. The method performed through injection molding or the method for performing stacking using a 3D printer or the like is preferable from the viewpoint of precisely finishing a complicated cross-sectional shape. Furthermore, the method performed through injection molding is particularly preferable from the viewpoints of manufacturing a stent at low cost and finishing the surface in a smooth state.

An operation of the stent of the present embodiment will be described.

The distal portion of the balloon catheter 10 is positioned at a lesion area, with the stent 20 positioned in covering and axial overlapping relation to the balloon 13, and a dilation fluid is introduced into the balloon 13 to dilate the balloon 13. The stent 20 is expanded in the radial direction R in accordance with the dilation of the balloon 13.

In the stent 20, the first annular portions 51 in which the struts 30 “densely” exists and the second annular portions 52 in which the struts 30 “sparsely” exists are regularly repeatedly arranged in the axial direction D1 (refer to FIG. 3). The amount of pinching of the balloon 13 by the struts 30 during crimping is larger in the second annular portion 52 in which the strut 30 “sparsely” exists than in the first annular portion 51 in which the strut 30 “densely” exists. Accordingly, when the balloon 13 is dilated, the second annular portions 52 of the stent 20 are more easily expanded than the first annular portions 51.

In a case where the amount of pinching of the balloon 13 by the struts is uniform in the axial direction D1, both end portions of the balloon 13 in the axial direction D1 are relatively easily dilated. Therefore, the stent is expanded such that both end portions of the stent in the axial direction D1 are first bulged, and then, a central portion is bulged in accordance with the bulging of the both end portions. In contrast, with the embodiment of the stent described above representing one example of the inventive stent disclosed here, where the amount of pinching of the balloon 13 by the struts 30 changes at regular sparseness and denseness in the axial direction D1 (i.e., the amount of pinching of the balloon by the struts 30 alternates between more sparse and less sparse (less dense and more dense) along the axial extent D1 of the stent), the stent 20 is expanded so as to be almost uniformly bulged in the axial direction D1, compared to a case where the pinching amount is uniform. As a result, the expansion state and the expansion timing of the stent 20 is uniform in the axial direction D1, and therefore, it is possible to suppress deviation of expansion of the stent 20 in the axial direction D1 during the expansion of the stent 20.

After the stent 20 is expanded, the first link portions 61 are coaxially positioned (on the first lines 71) and the second link portions 62 are coaxially positioned (on the second lines 72). For this reason, it is possible to secure the collapse strength in the axial direction D1, and therefore, the length variation (shortening) of the stent 20 during expansion in the axial direction D1 becomes almost zero. In addition, since the stent 20 has a high strength in the axial direction D1, it is difficult for the stent 20 to be deformed (deformation) in the axial direction D1 when delivering the stent 20 using the balloon catheter 10.

In the stent 20, the first annular portions 51 in which the struts 30 “densely” exist and the second annular portions 52 in which the struts 30 “sparsely” exist are regularly repeatedly arranged in the axial direction D1. In other words, since the first annular portions 51 which is relatively rigid (more rigid than the second annular portions 52) and the second annular portions 52 which is relatively flexible (more flexible than the first annular portions 51) are regularly repeatedly arranged in the axial direction D1, of the ability of the stent 20 to be passed through the lumen in the living body at a position of a curved lesion or the like becomes satisfactory. The shrinkage (recoil) in the circumferential direction D2 in the stent 20 during expansion is also reduced due to the first annular portion 51 which is relatively rigid.

In this manner, in the stent 20 of the present embodiment, the variation in the length of the stent during expansion becomes small and the stent has satisfactory flexibility so as to be able to pass through a biological lumen, and therefore, it is possible to accurately indwell the stent in a target lesion area.

An evaluation comparison test was performed on a test product of the stent 20 having the annular bodies 40 of which the number of mountains 31 of the first annular portion 51 is ten and the number of mountains 31 of the second annular portion 52 is eight, and an existing product of which the number of mountains 31 of all annular portions is eight. In the test product, the flexibility is excellent by about 10% to 12% due to two-point flexure compared to the existing product, the shortening becomes almost zero, and recoil is also slightly reduced.

In this manner, it is possible to check an effect obtained by the present embodiment in which the stent 20 is uniformly expanded by regularly making variation in the amount of pinching of the balloon 13 during crimping using the sparseness and denseness of the stent, and therefore, an improvement in the variation (shortening or recoil) in physical properties of the stent 20 after the expansion can be seen compared to the existing product.

As described above, according to the stent 20 of the present embodiment, the number of mountains 31 in the first annular portion 51 and the number of mountains 31 in the second annular portion 52 are different from each other between the first link portions 61 adjacent to each other in the circumferential direction D2. The first link portions 61 are positioned on the first lines 71 parallel to the axial direction D1 and the second link portions 62 are positioned on the second lines 72 parallel to the axial direction D1, in a state where the plurality of annular bodies 40 are repeatedly arranged in the axial direction D1.

According to such a configuration, it is possible to provide the stent 20 which has satisfactory flexibility so as to be able to pass through a biological lumen and in which an expansion state and an expansion timing of the stent 20 becomes uniform in the axial direction D1 and the variation of the stent during expansion is small, and therefore, it is possible to accurately indwell the stent in a target lesion area.

The first lines 71 on which the first link portions 61 are positioned and the second lines 72 on which the second link portions 62 are positioned are offset to each other in the circumferential direction D2.

According to such a configuration, when comparing to a different configuration in which the first lines 71 and the second lines 72 are coaxially positioned, twisting of the whole stent 20 in the circumferential direction D2 or deflection from the axial direction D1 is easily caused, and therefore, the flexibility of the stent 20 is improved. As a result, passing properties of the stent 20 at a position of a curved lesion or the like becomes more satisfactory.

The number of first link portions 61 in one annular body 40 and the number of second link portions 62 between the annular bodies 40 are equal to each other. Furthermore, the plurality of second link portions 62 connect the annular bodies 40 at a central position between the first link portions 61 adjacent to each other in the circumferential direction D2.

According to such a configuration, it is possible to uniformly secure the collapse strength of the stent 20 in the axial direction D1 in the circumferential direction D2 and to uniformly secure the flexibility of the stent 20 regardless of the position of the axial direction D1. As a result, the passing properties of the stent 20 at a position of a curved lesion or the like becomes more satisfactory while uniformly maintaining the high strength in the axial direction D1, in the circumferential direction D2.

The stent described above can be appropriately modified without being limited to the above-described embodiment.

An example of a combination of the number of mountains 31 being ten and eight in the first annular portion 51 and the second annular portion 52 included in the annular body 40 has been shown. However, the present invention is not limited to this case. For example, the combination of number of mountains 31 may be set to ten and six. In addition, it is also possible to set a combination of the number of mountains to be a multiple of the number of first link portions 61. The number of mountains 31 is preferably set to a multiple of the number of first link portions 61 in terms of obtaining excellent expansion uniformity.

The embodiment including two annular portions of the first annular portion 51 and the second annular portion 52 in the annular body 40 has been shown. However, the present invention is not limited to this case. For example, the present invention may include three or more annular portions and the number of mountains 31 in each of the annular portions may be different from the other. When combining the number of mountains 31, it is possible to appropriately select an arrangement way in a descending order such as ten-nine-eight, an arrangement way in an ascending order such as eight-nine-ten, an arrangement way in which the center is reduced, for example, ten-eight-ten, an arrangement way in which the center is increased, for example, eight-ten-eight, and the like as long as it is possible to arrange the strut 30 by regularly repeating the sparseness and denseness of the strut in the axial direction D1. The case where the amplitude A1 of the first annular portion 51 and the amplitude A2 of the second annular portion 52 are equal to each other has been shown, but may be different from each other.

The detailed description above describes a stent and operation of the stent. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent comprising:
a plurality of struts which together form a cylindrical circumference;
each strut possessing a wave shape in which a plurality of mountains are configured as convex portions that project toward both ends of the cylindrical shape in an axial direction;
annular bodies repeatedly arranged in the axial direction, each annular body including at least
a first annular portion comprised of one of the struts and possessing an annular shape,
a second annular portion comprised of another of the struts and possessing an annular shape, the second annular portion being positioned adjacent the first annular portion in the axial direction;
a plurality of first link portions disposed between the first annular portion and the second annular portion of each of the annular bodies, the first link portions of each annular body being circumferentially spaced from each other and connecting the first annular portion to the second annular portion;
a plurality of second link portions disposed between and connecting the annular bodies that are adjacent to each other in the axial direction, the second link portions connecting the axially adjacent annular bodies being circumferentially spaced from each other;
intervals between the first link portions adjacent to each other in the circumferential direction being equal;
intervals between the second link portions adjacent to each other in the circumferential direction being equal;
for each of the annular bodies, a total number of mountains in the first annular portion and a total number of mountains in the second annular portion between the circumferentially adjacent first link portions differing from each other; and
one of the first link portions of each annular body being positioned on one first line parallel to the axial direction, the other of the first link portions of each annular body being positioned on another first line different from the one first line and parallel to the axial direction, one of the second link portions between each pair of axially adjacent annular bodies being positioned on a second line parallel to the axial direction.

2. The stent according to claim 1, wherein the one first line, the other first line and the second line are offset from one another in the circumferential direction.

3. The stent according to claim 2, wherein the total number of the first link portions in one of the annular bodies and the total number of the second link portions between the axially adjacent annular bodies are equal, and wherein the plurality of second link portions connect the annular bodies at an axially central position between the circumferentially adjacent first link portions.

4. The stent according to claim 1, wherein the number of the first link portions in one of the annular bodies and the number of the second link portions between the axially adjacent annular bodies are equal, and wherein the plurality of second link portions connect the annular bodies at an axially central position between the circumferentially adjacent first link portions.

5. The stent according to claim 1, wherein each of the annular bodies is configured so that the first annular portion of one annular body is always positioned axially between the second annular portion of the one annular body and the second annular portion of the annular body that is axially adjacent the one annular body.

6. The stent according to claim 1, wherein there is a total of two of the first link portions disposed between the first annular portion and the second annular portion of each annular body.

7. A stent possessing a central axis and an axial extent, the stent comprising:
a plurality of annular bodies arranged in an axial direction along the axial extent to define a cylindrical shape with a plurality of through openings, each annular body comprising: a first annular portion comprised of a strut possessing an annular shape; and a second annular portion comprised of a strut possessing an annular shape, the second annular portion being positioned axially adjacent the first annular portion;
the strut comprising the first annular portion of each annular body being wavy-shaped in which a plurality of mountains project towards opposite axial ends of the stent;
the strut comprising the second annular portion of each annular body being wavy-shaped in which a plurality of mountains project towards the opposite axial ends of the stent;
each of the annular bodies comprising a plurality of circumferentially spaced apart first link portions connecting the strut of the first annular portion and the strut of the second annular portion, the circumferentially spaced apart first link portions of each annular body including one first link portion and an other first link portion;
each pair of axially adjacent annular bodies being connected to one another by at least one second link portion;
the plurality of annular bodies comprising a first annular body, a second annular body and a third annular body;
the first link portions of the first annular body being circumferentially spaced apart by a circumferential spacing that is the same as the circumferential spacing between the first link portions of the second annular body and that is also the same as the circumferential spacing between the first link portions of the third annular body;
for each of the first, second and third annular bodies, the total length of the strut of the first annular portion and the total length of the strut of the second annular portion differing from each other, the total length of the strut of the first annular portion being defined by the distance from a point on the strut of the first annular portion along the entire circumference of the strut of the first annular portion back to the point on the strut of the first annular portion, the total length of the strut of the second annular portion being defined by the distance from a point on the strut of the second annular portion along the entire circumference of the strut of the second annular portion back to the point on the strut of the second annular portion;
the one first link portion of the first annular body, the one first link portion of the second annular body and the one first link portion of the third annular body being aligned with one another and positioned along one first line parallel to the central axis;
the other first link portion of the first annular body, the other first link portion of the second annular body and the other first link portion of the third annular body being aligned with one another and positioned along an other first line parallel to the central axis and circumferentially spaced from the one first line; and
the second link portion of the first annular body, the second link portion of the second annular body and the second link portion of the third annular body being aligned with one another and positioned along a second line parallel to the central axis and circumferentially spaced from the one first line and the other first line.

8. The stent according to claim 7, wherein the one first line, the other first line and the second line are offset from one another in the circumferential direction.

9. The stent according to claim 7, wherein the second link portion connecting the axially adjacent annular bodies is located at an axially central position between the circumferentially adjacent first link portions.

10. The stent according to claim 7, wherein each of the annular bodies is configured so that the first annular portion of one annular body is always positioned axially between the second annular portion of the one annular body and the second annular portion of the annular body that is axially adjacent the one annular body.

11. The stent according to claim 7, wherein there is a total of two of the first link portions disposed between the first annular portion and the second annular portion of each annular body.

12. A method comprising:
inserting a stent into a living body, the stent being positioned on a balloon and comprising: a plurality of annular bodies arranged in an axial direction along the axial extent to define a cylindrical shape with a plurality of through openings, each annular body comprising a first annular portion comprised of a strut possessing an annular shape and a second annular portion comprised of a strut possessing an annular shape, the second annular portion being positioned axially adjacent the first annular portion; the strut comprising the first annular portion of each annular body and the strut comprising the second annular portion of each annular body being wavy-shaped in which a plurality of mountains project towards opposite axial ends of the stent, the strut comprising the first annular portion of each annular body being more dense than the strut comprising the second annular portion of each annular body; and
inflating the balloon to outwardly expand the second annular portion of the stent more easily than the first annular portion of the stent by virtue of the strut comprising the first annular portion of each annular body being more dense than the strut comprising the second annular portion of each annular body.

13. The method according to claim 12, wherein each of the annular bodies comprises a plurality of circumferentially spaced apart first link portions connecting the strut of the first annular portion and the strut of the second annular portion, the circumferentially spaced apart first link portions of each annular body including one first link portion and an other first link portion.

14. The method according to claim 13, wherein each pair of axially adjacent annular bodies is connected to one another by at least one second link portion.

15. The method according to claim 14, wherein the plurality of annular bodies comprise first, second and third annular bodies, the first link portions of the first annular body being circumferentially spaced apart by a circumferential spacing that is the same as the circumferential spacing between the first link portions of the second annular body and that is also the same as the circumferential spacing between the first link portions of the third annular body.

16. The method according to claim 12, wherein the first annular portion of each annular body is more dense than the strut comprising the second annular portion of the respective annular body by virtue of the total length of the strut of the first annular portion being greater than the total length of the strut of the second annular portion, the total length of the strut of the first annular portion being defined by the distance from a point on the strut of the first annular portion along the entire circumference of the strut of the first annular portion back to the point on the strut of the first annular portion, the total length of the strut of the second annular portion being defined by the distance from a point on the strut of the second annular portion along the entire circumference of the strut of the second annular portion back to the point on the strut of the second annular portion.

17. The method according to claim 12, wherein:
the plurality of annular bodies comprise first, second and third annular bodies
the first, second and third annular bodies each comprise a plurality of circumferentially spaced apart first link portions connecting the strut of the first annular portion and the strut of the second annular portion, the circumferentially spaced apart first link portions of each annular body including one first link portion and an other first link portion; and
the one first link portion of the first annular body, the one first link portion of the second annular body and the one first link portion of the third annular body being aligned with one another and positioned along one first line parallel to the central axis.

18. The method according to claim 17, wherein:
the other first link portion of the first annular body, the other first link portion of the second annular body and the other first link portion of the third annular body being aligned with one another and positioned along one first line parallel to the central axis.
